(19) United States
(12) Patent Application Publication
(10) Pub. No.: US 2010/0166777 A1
(43) Pub. Date: Jul. 1, 2010

(54) HEMATOPOIETIC CELLS THAT EXPRESS MOSC-1

(75) Inventors: Sergio Abrignani, Serre Di Rapolano (Siena) (IT); Mariacristina Crosti, Milano (IT); Monica Moro, Pioltello (Milano) (IT)

Correspondence Address: LUCAS & MERCANTI, LLP, 475 PARK AVENUE SOUTH, 15TH FLOOR, NEW YORK, NY, 10016, US

(73) Assignee: ISTITUTO NAZIONALE DI GENETICA MOLECOLARE-INGM, Milano (IT)

(21) Appl. No.: 12/601,474

(22) PCT Filed: May 27, 2008
(86) PCT No.: PCT/IB08/01326

§ 371 Date: Nov. 23, 2009

(30) Foreign Application Priority Data
May 28, 2007 (IT) MI2007A001084

(51) Int. Cl. A61K 35/14; C12N 5/078; C12Q 1/02; C07K 14/42; C07K 16/40; A61K 38/16; A61K 39/395; A61P 35/02

(52) U.S. Cl. 424/173.1; 435/372; 435/29; 424/93.7; 530/396; 530/389.6; 530/388.7; 530/391.7; 514/8

(57) ABSTRACT

The present invention relates to ex-vivo cells belonging to the hematopoietic system, characterized by the presence of MOSC-1 protein on cell surface, methods for isolating them and uses thereof.

The present invention relates to ex-vivo cells belonging to the hematopoietic system, characterized by the presence of MOSC-1 protein on cell surface, to methods for isolating them and the uses thereof. MOSC-1/mosc-1 gene is known at the state of the art thanks to its genic sequence. The gene for Homo sapiens is represented by GeneID no. 64757 according to designation (http://www.ncbi.nlm.nih.gov/entrez). MOSC-1 gene is present on chromosome 1 in 1q41 position. The acronym for mosc-1/MOSC-1 protein stands for MOCO sulphurase C-terminal domain containing 1: it is therefore a protein comprising MOCO protein domain (Molybdenum-containing cofactor). Various functions have been proposed for said protein, among which the function of oxidoreductase and/or transferase. It is further believed that the final position of MOSC-1 protein is in mytochondrial membranes because the sequence contains mytochondrial precursors. A discussion on possible MOSC-1 functions can be found in “MOSC domains: ancient, predicted sulfur-carrier domains, present in diverse metal-sulfur cluster biosynthesis proteins including Molybdenum cofactor sulfurases”, Vivel Anantharaman et al., FEMS 207 (2002), pp. 55-61. US 2006/0177814 demonstrates using micro-array technology that MOSC-1 was abnormally expressed in neutrophils of patients suffering from auto-immune disease Lupus.

At the state of the art there is the need to improve isolation and recognition procedures for specific cells belonging to the hematopoietic system and to improve the applications thereof in the therapeutic/diagnostic/prognostic field.

In the framework of the present invention, “hematopoietic system” refers to a group of cells present in a mammalian and evolving starting from hematopoietic stem cell (HSC) present in bone marrow, evolving according to dendrogram-like lineage up, to fully differentiated cells of the peripheral blood system. Moreover, at the state of the art there is the need to define the metabolic and/or physiologic status of a cell belonging to the hematopoietic system.

Surprisingly, the Applicant has found out that the above mentioned needs can be met exploiting the presence of MOSC-1 protein on the surface of specific cells belonging to the hematopoietic system.

The present invention is further disclosed below thanks to the accompanying drawings.

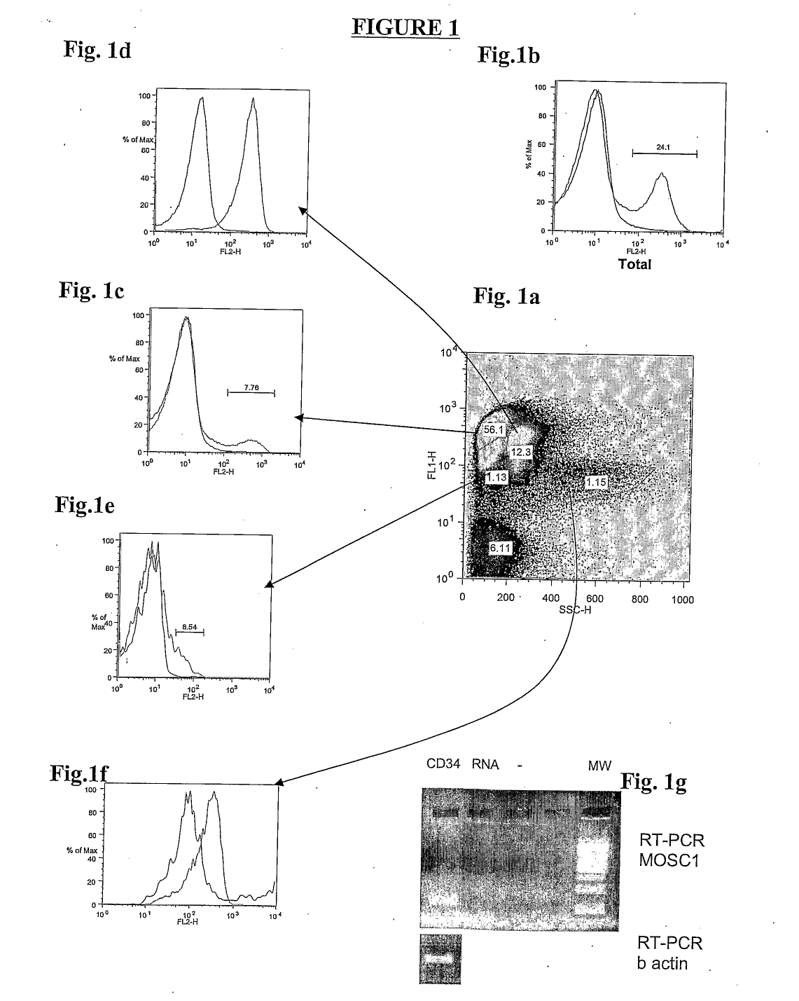

FIG. 1 shows the results of the expression of MOSC-1 on the surface of cells from umbilical cord blood.

FIG. 1a shows a cytometric analysis using FACS (ref. Example 1) according to physical parameters of granulosity (SSC) and fluorescence (FL-1) for CD45 so as to divide the different sub-populations of cells belonging to the hematopoietic system in umbilical cord blood.

FIG. 1b shows the expression of MOSC-1 on the surface of all populations of the hematopoietic system as detected using FACS (ref. Example 1). The number represents the percentage of cells expressing MOSC-1.

FIG. 1c shows the expression of MOSC-1 in the specific lymphocyte sub-population detected in FIG. 1a (ref. Example 1). The number represents the percentage of lymphocytes expressing MOSC-1.

FIG. 1d shows the expression of MOSC-1 in the specific monocyte sub-population detected in FIG. 1a (ref. Example 1). Note that the whole, or essentially the whole population expresses MOSC-1.

FIG. 1e shows the expression of MOSC-1 in the specific sub-population of hematopoietic stem cells detected in FIG. 1a (ref. Example 1). The number represents the percentage of hematopoietic stem cells representing MOSC-1.

FIG. 1f shows the expression of MOSC-1 in the specific myelocyte sub-population detected in FIG. 1a (ref. Example 1). Note that the whole, or essentially the whole population expresses MOSC-1.

FIG. 1g shows the results of a RT-PCR experiment (ref. Example 1) in which there is a high expression of MOSC-1 in cells expressing CD34. The expression of beta-actin is shown as experimental control.

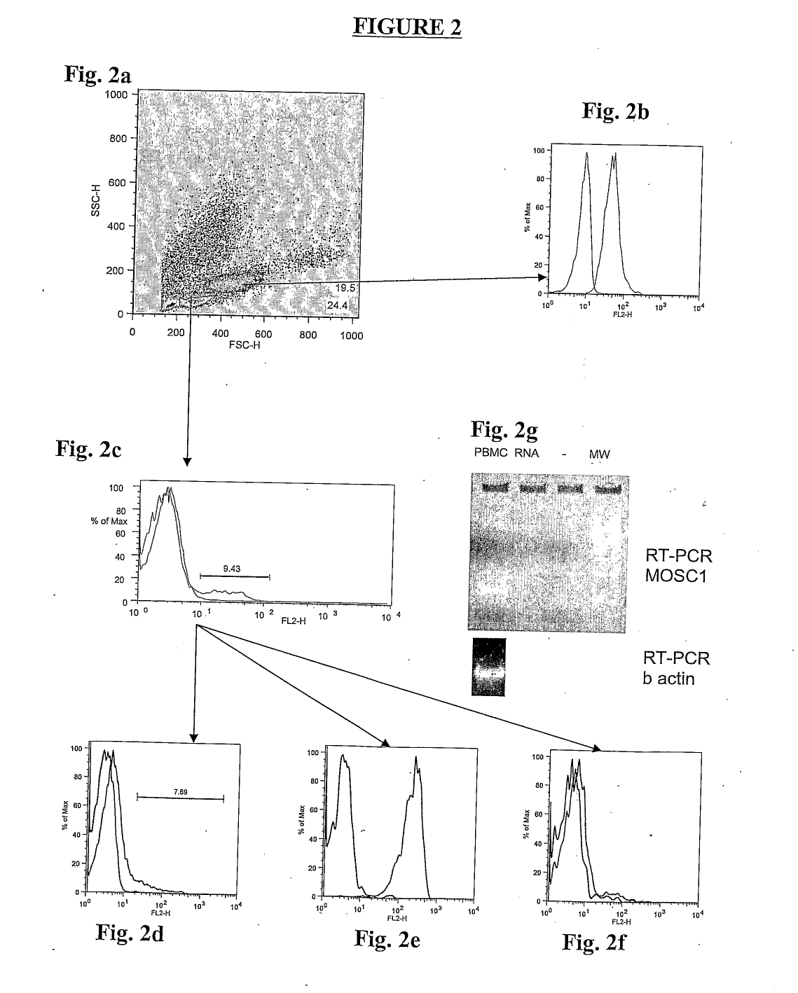

FIG. 2 shows the results of the expression of MOSC-1 on the surface of cells of human peripheral blood.

FIG. 2a shows a cytometric analysis using FACS (ref. Example 2) according to physical parameters of size (FSC) and granulosity (SSC) so as to divide lymphocytes and monocytes present in human peripheral blood.

FIG. 2b shows the expression of MOSC-1 in the specific monocyte sub-population detected in FIG. 2a (ref. Example 2). Note that the whole, or essentially the whole population expresses MOSC-1.

FIG. 2c shows the expression of MOSC-1 in the specific sub-population of all lymphocytes, detected in FIG. 2a (ref. Example 2). The number represents the percentage of all lymphocytes expressing MOSC-1.

FIG. 2d shows the expression of MOSC-1 in the specific T-lymphocyte sub-population detected in FIG. 2a (ref. Example 2) and FIG. 2c. The number represents the percentage of T-lymphocytes expressing MOSC-1.

FIG. 2e shows the expression of MOSC-1 in the specific B-lymphocyte sub-population detected in FIG. 2a (ref. Example 2) and FIG. 2c. Note that the whole, or essentially the whole population expresses MOSC-1.

FIG. 2f shows the expression of MOSC-1 in the specific sub-population of natural killer cells, detected in FIG. 2a (ref. Example 2) and FIG. 2c. The population does not express MOSC-1.

FIG. 2g shows the results of a RT-PCR experiment (ref. Example 2) in which there is a high expression of MOSC-1 in PBMC cells. The expression of beta-actin is expressed as experimental control.

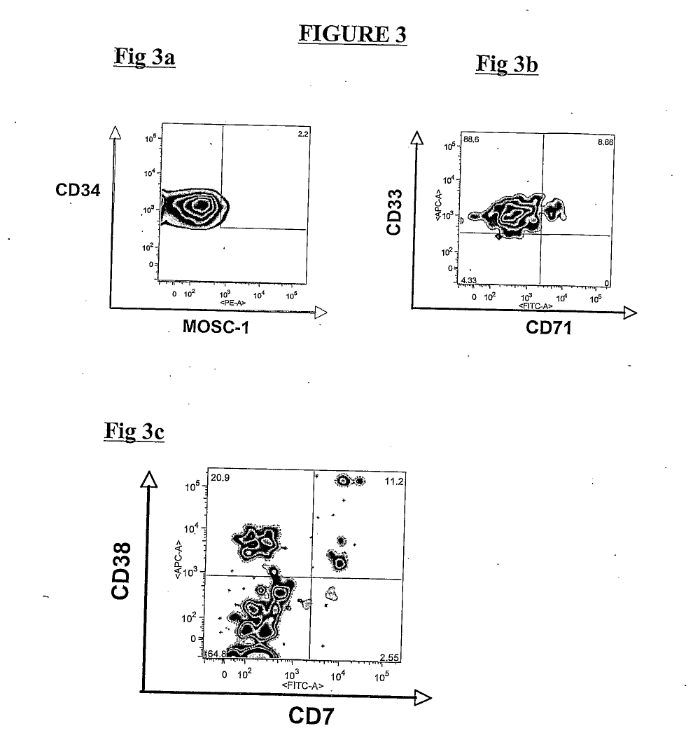

FIG. 3 shows the results of the expression of MOSC-1 on the surface of sub-populations of hematopoietic stem cells from umbilical cord blood (ref. Example 3).

FIG. 3a shows the percentage of HSCs expressing MOSC-1. The number in the top right corner represents the percentage of HSCs expressing MOSC-1.

FIG. 3b shows the percentage of HSCs expressing MOSC-1 and further expressing CD 33, a precursor marker for granulocytary/myelocytary cells. Note that essentially all cells express said marker. CD71 is an activity marker.

FIG. 3c shows the percentage of HSCs expressing MOSC-1 and further expressing CD38 and/or CD7, both known precursor markers for lymphocytary cells. Note that the percentage of HSCs expressing MOSC-1 that expresses said markers is relatively low.

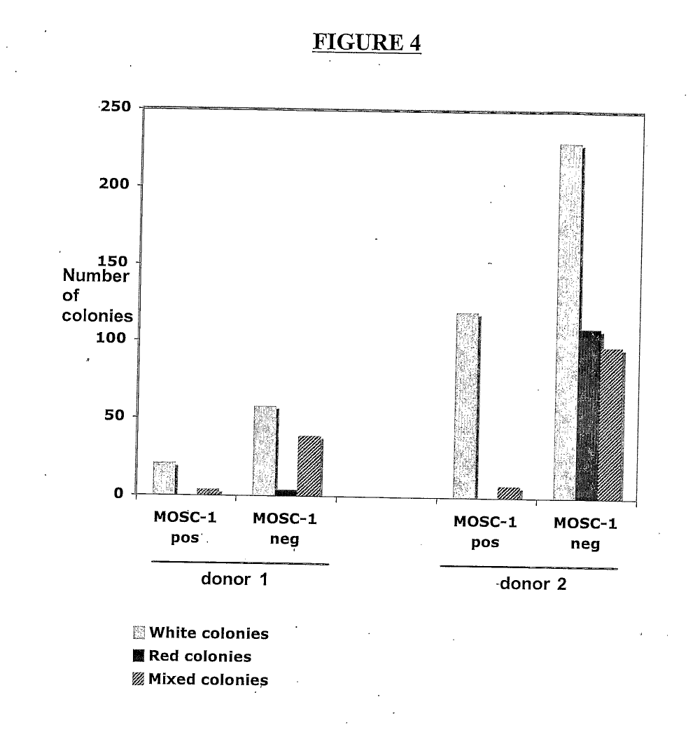

FIG. 4 schematically shows the results of a clonogenic assay on HSCs deriving from two donors (ref. Example 4). In both cases, cells grown from HSCs expressing MOSC-1 are compared to cells not expressing MOSC-1. Note that in both analyzed donors, HSCs expressing MOSC-1 mainly generate white colonies, whereas the number of red or mixed colonies is significantly lower than those generated by HSCs not expressing MOSC-1.

An object of the present invention consists of ex-vivo cells belonging to the hematopoietic system and characterized by the presence of MOSC-1 protein on cell surface.

In the framework of the present invention, “cells” refers to a number of cells comprising one or more cells.

In the framework of the present invention, the expression of a protein “on cell surface” refers to the expression of a protein getting through the membrane or anchored to the membrane or bound to the membrane or bound to a protein with the above mentioned characteristics, which shows anyhow at least part of its three-dimensional structure on the outer surface of cell membrane. The cells according to the invention preferably comprise hematopoietic stem cells (HSCs), lymphoid or myeloid common progenitors, proerythroblasts, erythroblasts, myeloblasts, lymphoblasts, monoblasts and mature leucocytes. The cells are more preferably HSCs and/or myeloid common progenitors, still more preferably HSCs. These cells are known at the state of the art since they are constituents of the hematopoietic system and distinguish according to methods known at the state of the art for their morphologic, genomic and proteomit characteristics. In the framework of the present invention, the various populations of cells belonging to the hematopoietic system, as described above and known at the state of the art, are distinguished one from the other with the term “sub-population”.

In the framework of the present invention, the term “cells” includes all maturation stages of said cell. For instance, the term “B-lymphocytes” includes all possible stages of B-lymphocytes from pro-B cells (CD34⁺CD19⁺CD20⁻Ig⁻) up to e.g. a plasma cell (CD38⁺CD27⁺CD19^{+/-}CD20⁻HLA⁻DR⁻).

The development stages according to the hematopoietic lineage of the cells according to the invention can be indicated by the positions of the cells in the organs and vessels of the hematopoietic system. The organs and vessels of the hematopoietic system are those known at the state of the art and include bone marrow, lymph nodes and blood or lymphoid vessels. The Applicant has found out that said cells according to the invention are present in the various districts of the blood system. Therefore, in the framework of the present invention, the cells according to the invention can derive from one or more hematopoietic systems with different or identical districts in the hematopoietic system.

The cells according to the invention preferably derive from humans. Humans can be at any development stage, such as e.g. an adult or a fetus. The cells according to the invention are present in all analyzed development stages of the organism. In the framework of the present invention, “cells from native populations” means the total group of cells, still belonging to the hematopoietic system, before the selection of cells expressing MOSC-1 on their surface. Said sample of native cells comprises both cells expressing MOSC-1 and those not expressing MOSC-1 on their surface. Said cell sample can derive from any cell source belonging to the hematopoietic system and known in the field, preferably an in-vivo source. Said source is preferably bone marrow, peripheral blood or umbilical cord blood.

According to a preferred aspect of the invention, the cells according to the invention comprise HSCs. Preferably, HSCs present in the cells according to the invention are 0.1 to 25% of native hematopoietic stem cells, more preferably 5 to 10% and still more preferably 8 to 9%. Still more preferably, HSCs are defined as cells expressing CD34⁺CD45^{dim} (in the frame-work of the present invention, ^{dim} means an intermediate expression level).

According to another aspect of the invention, the cells according to the invention comprise monocytes. Preferably, monocytes present in the cells according to the invention are 80 to 100% of native monocytes, more preferably 90 to 100% and still more preferably 95 to 100%. Still more preferably, monocytes are defined as cells expressing CD14 and defined according to cytometric parameters of size (FSC) and granulosity (SSC) known at the state of the art, as shown in FIG. 2a.

According to another aspect of the invention, the cells according to the invention comprise myelocytes. Preferably, myelocytes present in the cells according to the invention are 80 to 100% of native myelocytes, more preferably 90 to 100% and still more preferably 95 to 100%. Still more preferably, myelocytes are defined as cells expressing CD45^{dim} and having a high granulosity (SSC), as known at the state of the art and shown in FIG. 1a.

According to another aspect of the invention, the cells according to the invention comprise lymphocytes. Preferably, lymphocytes present in the cells according to the invention are 6 to 10% of native lymphocytes, more preferably 7.5 to 8%. Still more preferably, lymphocytes are defined as cells expressing CD45 high and according to cytometric parameters of size (FSC) and granulosity (SSC) as shown in FIG. 2a.

In a preferred embodiment of said aspect of the invention, lymphocytes are preferably divided. Preferably, the cells according to the invention comprise B-lymphocytes. Still more preferably, said B-lymphocytes present in the cells according to the invention are 80 to 100% of native B-lymphocytes, more preferably 90 to 100% and still more preferably 95 to 100%. Still more preferably, B-lymphocytes are defined as cells expressing CD19 in the region identifying lymphocytes. Similarly preferably, the cells according to the invention comprise T-lymphocytes. Still more preferably, said T-lymphocytes present in the cells according to the invention are 2.5 to 15% of native T-lymphocytes, more preferably 6 to 10% and still more preferably 7.5 to 8%. Still more preferably, T-lymphocytes are defined as cells expressing CD3 in the region identifying lymphocytes.

A further object of the invention consists of a method for selecting (identifying and/or isolating) the cells according to the invention, characterized by at least one step in which the presence of MOSC-1 on the surface of said cells is exploited.

Said method for selecting the cells according to the invention comprises the following steps:
preparing a sample of cells comprising hematopoietic cells,
determining the presence of MOSC-1 on the surface of the cells of said sample with a ligand for MOSC-1, and
isolating from the sample the cells on which MOSC-1 is present.

In a preferred embodiment, said method includes a step, before, during or after the step in which the presence of MOSC-1 is determined, in which the cells are selected positively or negatively for one or more specific sub-populations of cells belonging to the hematopoietic system, preferably for sub-populations of HSCs and/or myeloid common progenitors.

In the method for determining cells according to the invention, a ligand for MOSC-1 is preferably used, more preferably a protein ligand, such as e.g. an antibody or a lectin protein.

Therefore, a further object of the present invention consists of a ligand for MOSC-1, preferably an ex-vivo MOSC-1 specific ligand, preferably a polyclonal or monoclonal antibody against MOSC-1.

Among said ligands, the preferred one is a monoclonal antibody against MOSC-1. The monoclonal antibody can be prepared with methods known at the state of the art, such as e.g. recombination methods or such as e.g. a method exploiting Kohler and Midstein's technology. Said method preferably includes the following steps:
i) immunizing an animal having a spleen with MOSC-1 protein so as to induce an immune response, preferably together with an adjuvant;
ii) removing the spleen from the animal and treating it so as to obtain a suspension of intact cells, and isolating therefrom leucocytes, such as e.g. B-lymphocytes;
iii) forming a hybridoma, e.g. by fusion, from a leucocyte cell isolated from the suspension obtained in (ii) with an immortalized cell, such as e.g. cells from a myeloma lineage HGRP^{-/-};
iv) enriching the number of cells formed in (iii) with a suitable medium, such as e.g. a cell feeder layer;
v) selecting by a negative selection method cells that have formed a functioning hybridoma, such as e.g. growing the cells formed in (iii) on a HAT medium if using a myeloma HGRP^{-/-};
vi) isolating cells producing antibodies against MOSC-1 by methods known at the state of the art, such as e.g. using MOSC-1 bound to a marker, e.g. a probe;
vii) isolating and increasing the selected cells so as to produce monoclonal antibodies against MOSC-1.

Said ligands can be used in separation protocols known at the state of the art, e.g. magnetic separation or other methods. The method for selecting cells according to the invention or specific cell sub-populations can include both and/or negative selection protocols known at the state of the art.

A preferred protocol to be used in the selection of said sub-population is a flow cytometry protocol which succeeds in isolating the sub-population according to the invention discriminating between cells expressing or not expressing MOSC-1. Still more preferred is a selection protocol in which flow cytometry with fluorochromes (FACS®, Beckton-Dickinson), preferably as final step and/or after an enrichment protocol, such as e.g. with a protocol including the use of magnetic spheres with specific antibodies bound thereon is used.

Example 1 describes in detail an embodiment, as a mere non-limiting example, of a method for identifying various sub-populations belonging to the hematopoietic system of cells according to the invention starting from blood taken from the umbilical cord.

Example 2 describes in detail an embodiment, as a mere non-limiting example, of a method for identifying various sub-populations belonging to the hematopoietic system of cells according to the invention starting from peripheral blood taken from adult humans.

A further object of the present invention consists of the use of the cells as described below.

In a first embodiment, the cells according to the invention can be used for assessing in vitro the effect of a biological sample or of an active substance on the growth and maturation of granulocytary or myelocytary cells. In particular, an object of the present invention consists of a method for assessing said effect, said method comprising the following steps:
assessing the number of HSCs and/or myeloid common progenitors expressing MOSC-1 present in a cell sample comprising hematopoietic cells,
contacting a biological sample or an active substance with said cell sample comprising hematopoietic cells,
assessing the number of HSCs and/or myeloid common progenitors expressing MOSC-1 after adding said biological sample or said active substance, and
assessing the effect deriving from the addition of said biological sample or said active substance on the growth and/or maturation of granulocytary or myelocytary cells.

The number of cells expressing MOSC-1 is preferably the number of HSCs expressing MOSC-1.

The contacting procedure for a biological sample or an active substance can vary depending on the requirements of the methods and can be suitably chosen by the skilled technician.

The method for assessing the number of HSCs and/or myeloid common progenitors expressing MOSC-1 before and after the addition preferably involves the ligand for MOSC-1 and can involve protocols as described above.

The biological sample can include new or known proteins or other types of molecules deriving from humans. The active substance is preferably a drug.

In another embodiment, the cells according to the invention can be used in vitro to diagnostic or prognostic purposes. The prognostic or diagnostic results are preferably related to the presence of MOSC-1 on cell surface.

In another embodiment, the cells according to the invention can be used as a drug.

In a preferred embodiment of said use as a drug, a sub-population of cells belonging to the hematopoietic system according to the invention comprising HSCs, preferably a sub-population of HSCs as mentioned above, is used for preparing a drug for the prophylaxis or stop or treatment of diseases in which MOSC-1 gene is not in functioning form or “wild-type”. In the preparation of said drug, cell transfusion aspects should be taken into consideration, e.g. the autologous nature (defined as people having cells with identical HLAs, human leucocyte antigens) of the donor's cells with respect to the recipient's.

In another preferred embodiment of said use as a drug, a sub-population of cells belonging to the hematopoietic cells according to the invention comprising HSCs, preferably a sub-population of HSCs as mentioned above, and/or comprising myeloid progenitor cells is used as a drug for increasing, or alternatively for preparing a drug for increasing, one or more populations of cells belonging to a granulocytary or myelocytary hematopoietic lineage. Increasing means also restoring cell population to levels that can be commonly accepted as a standard. Said one or more population of cells belonging to a granulocytary or myelocytary hematopoietic lineage are preferably granulocytes, monocytes or karyocytes.

Said uses for preparing a drug for increasing cell populations are applied according to methods known at the state of the art for cell transfusion to a patient. Said application can take place after a myelo- or lympho-ablative treatment, such as e.g. radiotherapy, after diseases such as e.g. leukemia.

In an embodiment of said use, said increase of one or more cell populations is part of a treatment or prophylaxis for granulocytopenia, thrombocytopenia, agranulocytosis or neutropenia.

The administration of the drugs in the framework of the present invention occurs through methods known at the state of the art, preferably by intravenous injection or direct injection into bone marrow.

The drugs prepared according to the invention can further comprise excipients and/or stabilizers and/or carriers.

In another embodiment, ex-vivo cells expressing MOSC-1 can be used for assessing the metabolic status of cells belonging to the hematopoietic system and preferably of cells expressing MOSC-1. In said use, the number of cells expressing MOSC-1 and the amount of MOSC-1 expressed on each cell can give a hint of a metabolic status of cells belonging to the hematopoietic cells and preferably of cells expressing MOSC-1. This metabolic status can be related to the cell metabolism of at least one of the following compounds: carbohydrates, polysaccharides, nucleotides, amino acids, lipids, co-factors and vitamins, secondary metabolites, ATP. This embodiment can be applied to cells belonging to the whole hematopoietic system or only to one or more specific sub-populations of cells belonging to the hematopoietic system as described above. The number of cells expressing MOSC-1 and the amount of MOSC-1 expressed on each cell can be assessed with methods known at the state of the art, e.g. through a ligand as described above. Among said ligands a monoclonal antibody is preferred, more preferably with a probe bound thereto, such as e.g. by way of a secondary antibody, so as to quantify the number of antibodies present and adhering to the cell.

In another embodiment, the presence of MOSC-1 on cell surface can be exploited for identifying the passage and the outcome of said cells through the hematopoietic lineage and/or for isolating said cells. Said passage through the hematopoietic lineage depends on the type of cell being examined and it is therefore related to its outcome. Hematopoietic lineages known at the state of the art can be divided into karyocyte, erythrocyte, myelocyte, lymphocyte and monocyte lineages.

The presence of MOSC-1 on cell surface further enables to assess the lineage to which the cell is dedicated even before said lineage has been undertaken by the cell. This is advantageous especially for the cells according to the invention comprising HSC. From said HSC it can be assesses, by exploiting the presence of MOSC-1 on the surface, whether the cell becomes a lymphoid or myeloid common progenitor, even before the HSC has undertaken said development. If the HSC expresses MOSC-1, said HSC will become a myeloid common progenitor and then turn into granulocytary or myelocytary cells.

The presence of MOSC-1 on cell surface is preferably assayed with a ligand as described above. Among said ligands a monoclonal antibody is preferred, still more preferably a monoclonal antibody with a marker bound thereto, such as e.g. by way of a secondary antibody, so as to quantify the number of antibodies present and bound to the cell.

A further object of the invention consists of the ex-vivo ligand for MOSC-1 protein and the uses thereof.

The ligand according to the invention is preferably proteic and still more preferably an antibody or a lectin protein against MOSC-1. Said antibody is monoclonal or polyclonal, preferably monoclonal. Said antibody can be synthesized according to methods known at the state of the art as described above.

The ligand according to the invention is preferably present in a composition. Said composition preferably comprises excipients and/or adjuvants and/or stabilizers and/or carriers and can be formulated according to methods known at the state of the art. The choice of these excipients and/or adjuvants and/or stabilizers and/or carriers in the composition varies depending on the use thereof, provided that it allows to keep the ligand suitable.

The ligand according to the invention can be used as a drug. Preferably, said ligand can be used for preparing a drug to be used in a diagnostic or prognostic assay so as to assess physiologic or molecular aspects involving the sub-population of cells belonging to the hematopoietic system and expressing MOSC-1. Said diagnostic assay can be either ex vivo or in vivo. In a preferred embodiment, said ligand is bound to a marker, such as e.g. a secondary antibody associated to a probe, such as e.g. a fluorescent, phosphorescent or radioactive probe, bound on the secondary antibody.

In another embodiment, the ligand according to the invention can be used for preparing a drug for qualitatively or quantitatively assessing the metabolic status, as described above, of cells belonging to the hematopoietic system. Said assessment of the metabolic status of cells belonging to the hematopoietic system can occur either ex vivo or in vivo.

In another preferred embodiment, said ligand can be used for preparing a drug for modulating the displacement of the cells according to the invention through a human body. Said displacement can be modulated under normal physiologic conditions or can be caused by an immune response.

In another embodiment of the invention, said ligand according to the invention can be used as a drug for, or alternatively in the preparation of a drug for, the prophylaxis or stop or treatment of diseases involving cells expressing MOSC-1 and belonging to the hematopoietic system. In this case, involved cells are preferably leucocytes and therefore diseases are of auto-immune type, such as e.g. non-Hodgkin lymphoma or Lupus. In said cases, the goal is to eliminate aberrantly working and noxious leucocytes. Preferably, cells are granulocytary or myelocytary and therefore the disease is acute myeloid leukemia or chronic myeloid leukemia. Thus the use of antibodies, preferably monoclonal antibodies, against MOSC-1 is preferred, since these can start an autologous ADCC or CDC cascade so as to eliminate the leucocytes they identify.

Preferably, the drug contains an adjuvant apt to induce an immune response.

In another embodiment, the ligand against MOSC-1, preferably an antibody, preferably a monoclonal antibody, is prepared with a noxious substance bound to the ligand, such as e.g. through a secondary antibody. Said noxious substance is toxic or anyhow apt to eliminate the ligand's goal, i.e. the cell expressing MOSC-1 on its surface. Said toxic substance can be a toxin or a radioactive atom, such as e.g. iodine-131 or an enzyme that might be later involved in a monoclonal therapy system known in the art as ADEPT. Said ligand can also be used as a drug for, or alternatively in the preparation of a drug for, the prophylaxis or stop or treatment of diseases involving cells expressing MOSC-1 and belonging to the hematopoietic system, as already described above.

### EXAMPLE 1

### Isolation of Sub-Populations of Cells According To the Invention Expressing MOSC-1 in Umbilical Cord Blood

Isolation of Mononucleated Cells from Blood

1. A bag of umbilical cord blood (75 ml) was obtained from Milano Cord Blood Bank and diluted 1:3 in a phosphate buffered saline (PBS) containing 2 mM ethylene diamine tetraacetic acid (EDTA).

2. 15 ml Ficoll-Hypaque (density 1.077 g/l) were introduced into a Falcon 50 ml tube, then 30 ml blood from umbilical cord were laid thereupon. Blood was let flow down very slowly so as not to perturb the interface. The operation was repeated until the whole sample was over.

3. The Falcon tube was then centrifuged at 1600 rpm for 30 min at room temperature, without brake. Mononucleated cells (CBMCs) placed themselves on the interface between Ficoll-Hypaque and plasma. Said CBMC ring was collected and transferred into a Falcon 50 ml tube.

4. CBMCs were washed once with 50 ml PBS containing 2 mM EDTA and with 5%; normal human serum (NHS) centrifuging for 10 min at 1200 rpm.

5. The pellet was then washed with 50 ml PBS 5% NHS centrifuging for 10 min at 1200 rpm, and washed again with 50 ml PBS 5% NHS centrifuging for 10 min at 800 rpm.

6. CBMCs contained in a pellet at the end of step 5 were resuspended in 10-30 ml PBS 5%, NHS at room temperature.

Isolation of Cells According to the Invention from Blood

7. Cells were counted with a Burker's chamber and 3×10⁶-5×10⁶ CBMCs pro sample were stained.

8. Samples were incubated for 20 min at room temperature with PBS 50% NHS.

9. Samples were centrifuged for 3 min at 1500 rpm and were incubated unwashed for 10 min in an ice bath with MOSC-1 antiserum diluted 1:50 in 100 microliters PBS 5% NHS.

MOSC-1 antiserum was prepared according to methods known at the state of the art, immunizing mice with MOSC-1 primary structure.

Samples for negative control were incubated for 10 min in ice with antiserum of a non-immunized mouse for setting the negativity of the final staining of the image resulting from FACS.

10. Cells of centrifuged samples were washed twice with PBS 5% NHS, removing the supernatant after centrifugation for 3 min at 1500 rpm and resuspending with PBS 5% NHS.

11. Said resuspended cells were then incubated again for 10 min in an ice bath with Goat-anti-mouse IgG-PE (Southern Biotech®), a known “secondary” antibody with fluorochrome phycoerythrine (PE), bound thereon, diluted 1:100 in 100 microliters PBS 5% NHS.

12. Cells were washed twice with PBS 5% NHS, centrifuging for 3 min at 1500 rpm and resuspending with PBS 5% NHS.

13. The resuspended pellet was added with 12 micrograms pro sample of mIgG (mouse immunoglobulines) and incubated for at least 60 min in ice.

14. Cells were then incubated again for 10 min in an ice bath with mouse-anti-hCD34PC5 (Beckman Coulter®), a known monoclonal antibody with fluorochrome PE-Cy5 bound thereon, and with mouse-anti-hCD45FITC (BD Biosciences), a known monoclonal antibody with fluorochrome fluorescein (FITC) bound thereon.

15. Eventually, stained cells were washed (centrifuging at 1500 rpm for 3 min) with PBS 10% NHS and resuspended in 500 microliters for acquisition with FAC-SCalibur®.

16. The BecktonDickinson-FACS® machine was operated in compliance with protocols known in the field and mentioned in Current Protocols in Immunology (2001), John Wiley and Sons Inc., Units 5.4.1-5.4.22 so as to obtain the results as shown in Table 1. Exposed fluorescences and resulting figures as shown in FIGS. 1b-1f are also obtained from FACSCalibur®.

In order to check the indications on the expression of MOSC-1 obtained from FACS experiment as disclosed above, a control experiment with RT-PCR was carried out so as to monitor the expression of MOSC-1 gene in CD34 expressing cells.

To this purpose, cells purified with Ficoll were used for enriching hematopoietic stem cells by means of specific antibodies conjugated with magnetic spheres (Miltenyi Biotech, cat# 130-046-702) in compliance with the supplier's protocol.

RNA was extracted from the cells obtained after enrichment, using Qiagen kit (cat# 74104) in compliance with the supplier's protocol, and cDNA was produced starting from 100 ng RNA using RetroScript enzyme (Ambion, cat# 1710) in compliance with the supplier's protocol.

2 μl cDNA were used for the analysis with RT-PCR, by means of MOSC-1 specific primers. RT-PCR for beta-actin gene was executed as positive control, since beta-actin is known to be a protein expressed by all cells. Primers that were used were the following:

### MOSC-1 fw: SEQ ID NO 1

### MOSC-1 rev: SEQ ID NO 2

Beta-actin gene fw: SEQ ID NO 3
Beta-actin gene rev: SEQ ID NO 4

The complete sequences are listed in the attachment in compliance with WIPO standard ST. 25 developed with software Patent-In 3.5. Conditions applied for RT-PCR

MOSC-1 specific primers were the following:

cDNA: 2 microliters
MOSC1 fw (10 microM): 1 microliter
MOSC1 rev (10 microM): 1 microliter
2×Taq PCR Master Mix (Qiagen, cat #201443): 25 microliters
Sterile water: up to a final volume of 50 microliters.

Conditions of PCR thermal cycles:

### 94° C., 3 min

30 cycles at 94° C. for 30 sec, 55° C. for 30 sec and 72° C. for 30 sec

### 72° C., 10 min

### ∞, 4° C.

Results are shown in FIG. 1g, where an expression of MOSC-1 gene and of control (gene for beta-actin) in cells expressing CD34 can be clearly recognized.

### EXAMPLE 2

### Isolation of Sub-Populations of Cells According to the Invention Expressing MOSC-1 in Human Peripheral-Blood

Isolation of mononucleated cells from blood

1. A 10 ml sample of peripheral blood from a healthy donor was diluted 1:3 in a phosphate buffered solution (PBS).

2. 15 ml Ficoll-Hypaque (density 1.077 g/l) were introduced into a Falcon 50 ml tube, then 30 ml peripheral blood from a healthy donor were laid thereupon. Blood was let flow down very slowly so as not to perturb the interface. The operation was repeated until the whole sample was over.

3. The Falcon tube was then centrifuged at 1600 rpm for 30 min at room temperature, without brake. Mononucleated cells (PBMCs) placed themselves on the interface between Ficoll-Hypaque and plasma. Said PCBMC ring was collected and transferred into a Falcon 50 ml tube.

4. PBMCs were washed twice with 50 ml PBS containing 5% normal human serum (NHS) centrifuging for 10 min at 1200 rpm.

5. The pellet was then washed with 50 ml PBS 5% NHS centrifuging for 10 min at 800 rpm.

6. PBMCs contained in a pellet at the end of step 5 were resuspended in 10-30 ml PBS 5% NHS at room temperature.

Isolation of Cells According to the Invention from Blood

A protocol similar to Example 1 (steps 7-16) was followed for isolating the cells according to the invention from the population of PBMCs prepared as described above.

Only step 14 was changed. At step 14 cells were incubated for 10 min in an ice bath with m-apha-hCD19Cychrome (BD Biosciences®), a known monoclonal antibody with fluorochrome PE-Cy5 bound thereon, and with mouse-anti-hCD3FITC (BD Biosciences), a known monoclonal antibody with fluorochrome fluorescein (FITC) bound thereon.

The BecktonDickinson-FACS® machine was operated in compliance with protocols known in the field and mentioned in Current Protocols in Immunology (2001), John Wiley and Sons Inc., Units 5.4.1-5.4.22 so as to obtain the results as shown in Table 2. Exposed fluorescences and resulting figures as shown in FIGS. 1b-1f are also obtained from FACSCalibur®.

In order to check the indications on the expression of MOSC-1 obtained from FACS experiment as disclosed above, a control experiment with RT-PCR was carried out so as to monitor the expression of MOSC-1 gene in PBMCs. The same protocol as in Example 1 was followed. The results are shown in FIG. 2g, where an expression of MOSC-1 gene and of control beta-actin PBMCs can be clearly recognized.

### EXAMPLE 3

### Assessment Of Lineage-Indicating Markers on Hematopoietic Stem Cells According to the Invention Expressing MOSC-1 in Umbilical Cord Blood

The assay as described in Example 1 was repeated, wherein the following reagents were introduced at step 14 so as to assess the presence of the following markers on HSCs:
antibody anti CD33-APC (BD Biosciences), a known monoclonal antibody conjugated with fluorochrome allophycocyanin,
antibody anti CD71-FITC (Immunotools), a known monoclonal antibody conjugated with fluorochrome fluorescein,
antibody anti CD7-FITC (BD Biosciences), a known monoclonal antibody conjugated with fluorochrome fluorescein,
antibody anti CD38 PE-Cy5 (BD Biosciences), a known monoclonal antibody conjugated with fluorochrome PE-Cy5.

Exposed fluorescences and resulting figures as shown in FIGS. 3a-3c were measured with FACScanto® machine. The results in FIG. 3 show that a small percentage of CD34⁺ cells expresses MOSC-1, but essentially whole said population expresses CD33. The results in FIG. 3c indicate that a small percentage of said CD33⁺ population expresses markers for lymphocytary lineages (CD38 and/or CD7).

### EXAMPLE 4

### Clonogenic Assay for Determining how HSCs Expressing MOSC-1 Mature

A clonogenic assay was executed using growth medium Methocult (Stem Cell Technology, cat. # 04433) in compliance with the manufacturer's protocol.

HSCs expressing MOSC-1 and HSCs not expressing MOSC-1, prepared and marked as described in Example 1, are separated by Fluorescent Activated Cell Sorting (FACS) using FACSaria® separator. The BecktonDickinson-FACS® machine was operated in compliance with protocols known in the field and mentioned in Current Protocols in Immunology (2001), John Wiley and Sons Inc.

Populations thus obtained have a purity typically above 99%.

200-500 cells for each population are inoculated into 5 ml growth medium enriched with methyl cellulose and Methocult growth factors (Stem Cell Technology, cat. # 04433).

The medium containing the cells is then plated in 35 mm Petri dishes.

Petri dishes are incubated at 37° C. in the presence of 5% CO₂ for 15 days.

After 15 days grown colonies are counted and assessed. The obtained results are shown in FIG. 4, where it can be clearly seen that hematopoietic stem cells expressing MOSC-1 mainly generate white colonies, whereas hematopoietic stem cells not expressing MOSC-1 generate all expected types of colonies.

1. Ex-vivo cells belonging to the hematopoietic system, characterized by the presence of MOSC-1 protein on cell surface.

2. The cells according to claim 1, wherein the cells belonging to the hematopoietic system are HSCs and/or myeloid common progenitors, preferably HSCs.

3. A method for selecting the cells according to claim 1, said method comprising the following steps:
preparing a sample of cells comprising hematopoietic cells,
assessing the presence of MOSC-1 on the surface of the cells of said sample with a ligand for MOSC-1, and
isolating from the sample the cells on which MOSC-1 is present.

4. The method according to claim 3, further comprising a step in which HSCs and/or myeloid common progenitors are isolated after preparing the sample comprising hematopoietic cells.

5. A method for assessing in vitro the effect of a biological sample or of an active substance on the growth and maturation of granulocytary or myelocytary cells, said method comprising the following steps:
assessing the number of cells according to claim expressing MOSC-1 present in a cell sample comprising hematopoietic cells,
contacting a biological sample or an active substance with said cell sample comprising hematopoietic cells,
assessing the number of cells according to claim 1 expressing MOSC-1 after adding said biological sample or said active substance, and
assessing the effect deriving from the addition of said biological sample or said active substance on the growth and/or maturation of granulocytary or myelocytary cells.

6. A drug containing the cells according to claim 1.

7. A method of increasing one or more populations of cells belonging to a granulocytary or myelocytary hematopoietic lineage, comprising injecting the cells according to claim 1 into the bone marrow of a human in need thereof.

8. The method according to claim 7, wherein said one or more populations of cells belonging to a granulocytary or myelocytary hematopoietic lineage are granulocytes, monocytes or karyocytes.

9. The method according to claim 7, wherein the increase of said one or more populations is part of a treatment or prophylaxis for granulocytopenia, thrombocytopenia, agranulocytosis or neutropenia.

10. A specific ligand for MOSC-1 protein.

11. The ligand according to claim 10, wherein the ligand is an antibody or a lectin against MOSC-1.

12. The ligand according to claim 11, wherein the ligand is an antibody, or a monoclonal antibody.

13. The ligand according to claim 10, wherein the ligand is bound to a noxious substance.

14. A drug containing the ligand according to claim 10.

15. The ligand according to claim 10 for use as a drug in A method for the prophylaxis or treatment of diseases involving cells expressing MOSC-1 and belonging to the hematopoietic system, comprising administering the ligand of claim 10 to a patient in need thereof.

16. The ligand method according to claim 15, wherein said diseases are acute myeloid leukemia or chronic myeloid leukemia.


SEQUENCE LISTING (4 sequences)

SEQ ID NO: 1 - DNA, 21 bp, Unknown
  Primer (ref. Example 1)
ttcctgaagtcacagccctac

SEQ ID NO: 2 - DNA, 21 bp, Unknown
  Primer (ref. Example 1)
gcatctggaacaagccatcac

SEQ ID NO: 3 - DNA, 17 bp, Unknown
  Primer (ref. Example 1)
tccagcgtactccaaag

SEQ ID NO: 4 - DNA, 17 bp, Unknown
  Primer (ref. Example 1)
cttgggctgtgacaaag